(19) United States
(12) Patent Application Publication
(10) Pub. No.: US 2022/0257167 A1
(43) Pub. Date: Aug. 18, 2022

(54) Biosensor

(71) Applicant: Nippon Telegraph and Telephone Corporation, Tokyo (JP)

(72) Inventors: Hiroto Matsuoka, Tokyo (JP); Takako Ishihara, Tokyo (JP); Hiroyoshi Togo, Tokyo (JP)

(21) Appl. No.: 17/596,074

(22) PCT Filed: Jun. 4, 2019
(86) PCT No.: PCT/JP2019/022153

§ 371 Date: Dec. 2, 2021

(51) Int. Cl. A61B 5/257; A61B 5/27; A61B 5/274; A61B 5/308; A61B 5/318; A61B 5/28

(52) U.S. Cl. CPC A61B 5/257; A61B 5/27; A61B 5/274; A61B 2560/0214; A61B 5/318; A61B 5/28; A61B 5/308

(57) ABSTRACT

In an embodiment, a biometric sensor includes: a substrate; an electrode connected to the substrate, the electrode configured to detect a biometric signal from a living body; a signal processor mounted on the substrate, the signal processor configured to process the biometric signal detected by the electrode to extract biometric information of the living body; a first wiring mounted on the substrate, the first wiring configured to send the biometric signal detected by the electrode to the signal processor; a power supply mounted on the substrate, the power supply configured to supply power to the signal processor; a second wiring mounted on the substrate, the second wiring configured to supply the power from the power supply to the signal processor; and an adhesive member on at least a portion of surfaces of the electrode and the substrate that adhere to clothes to be worn by the living body.

### CROSS-REFERENCE TO RELATED APPLICATION

This application is a national phase entry of PCT Application No. PCT/JP2019/022153, filed on Jun. 4, 2019, which application is hereby incorporated herein by reference.

### TECHNICAL FIELD

The present invention relates to a biometric sensor that adheres to clothes by for example ironing to acquire biometric information such as a person's electrocardiogram or heartbeat.

### BACKGROUND

The measurement of an electrocardiogram, a heartbeat, or the like is effective not only in the detection of a heart disease but also in a wide range of fields such as heat stroke prevention, management of physical conditions such as central fatigue detection and drowsiness detection, and heart rate training in sports and the like. As a means for easily measuring an electrocardiogram or a heartbeat, there is a wearable electrode inner wear using a conductive fabric “hitoe” (trade name) capable of measuring an electrocardiogram or a heartbeat just by being worn (see, for example, Non Patent Literature 1).

The wearable electrode inner wear of Non Patent Literature 1 is a wearable electrode inner wear that includes an electrode for detecting an electrocardiogram on the rear surface of a shirt, measures an electrocardiac signal with a transmitter mounted on the shirt, and transmits the electrocardiogram or heart rate to a smartphone or the like. Typically, in a case where an electrocardiogram is measured, a doctor or a laboratory technician needs to make an electrode adhere to a predetermined location and measure the electrocardiogram. However, such a shirt allows the electrode to be attached to an appropriate position on a user's body just by being worn, so that the user can easily measure an electrocardiogram or a heartbeat, and can easily use various services utilizing the measurement values.

### CITATION LIST

### Non Patent Literature

Non Patent Literature 1: Tsukada, Kasai, Kawano, Takagawara, Fujii, and Sumitomo: “Wearable Electrode Inner Wear That Measures Electrocardiogram Just by Being Worn” NIT Technical Journal, Vol. 26, No. 2, pp. 15-18, 2014

### SUMMARY

### Technical Problem

However, in order to measure an electrocardiogram or a heartbeat with a wearable electrode inner wear, it is necessary to wear a special shirt which is provided by a manufacturer of the wearable electrode inner wear, and a user cannot measure the electrocardiogram or heartbeat while wearing clothes that he/she has selected. In addition, the manufacturer is also required to perform many processes such as attachment of an electrode, wiring, attachment of a connection terminal to a transmitter, and waterproofing in order to manufacture the wearable electrode inner wear, which increases manufacturing costs and makes it difficult to deploy a wide variety of products.

The present invention has been made in view of the above problem, and an object thereof is to provide clothes provided with a biometric sensor having a low manufacturing cost which allows clothes selected by a user to easily measure an electrocardiogram or a heartbeat.

### Means for Solving the Problem

In order to achieve the above object, according to an embodiment of the present invention, there is provided a biometric sensor configured to acquire biometric information of a living body by adhering to clothes to be worn by the living body, the biometric sensor comprising: a substrate to which an electrode configured to detect a biometric signal from the living body is connected, and on which a signal processing unit configured to process the biometric signal detected by the electrode to extract the biometric information, a first wiring configured to send the biometric signal detected by the electrode to the signal processing unit, a power supply configured to supply power to the signal processing unit, and a second wiring configured to supply the power are mounted, wherein at least a portion of surfaces of the electrode and the substrate that adhere to the clothes is provided with an adhesive member for adhering to the clothes.

### Effects of Embodiments of the Invention

According to embodiments of the present invention, it is possible to provide clothes provided with a biometric sensor having a low manufacturing cost which allows clothes selected by a user to easily measure an electrocardiogram or a heartbeat.

### BRIEF DESCRIPTION OF THE DRAWINGS

FIG. 1A illustrates a configuration example of a surface (non-adhesive surface) of a biometric sensor according to an embodiment of the present invention.

FIG. 1B illustrates a configuration example of a rear surface (adhesive surface) of the biometric sensor according to the embodiment of the present invention.

FIG. 1C is a cross-sectional view of a substrate of the biometric sensor according to the embodiment of the present invention.

FIG. 1D is a cross-sectional view of an electrode of the biometric sensor according to the embodiment of the present invention.

FIG. 2 illustrates a configuration example of clothes (rear surface) to which the biometric sensor according to the embodiment of the present invention adheres.

FIG. 3 illustrates a configuration example of a connecting portion between the substrate and the electrode of the biometric sensor according to the embodiment of the present invention.

FIG. 4A illustrates a configuration example of an engagement portion between the substrate and a battery of the biometric sensor according to the embodiment of the present invention.

FIG. 4B illustrates another configuration example of the engagement portion between the substrate and the battery of the biometric sensor according to the embodiment of the present invention.

FIG. 5 illustrates a configuration example of a rectifier circuit which is disposed between the substrate and the battery of the biometric sensor according to the embodiment of the present invention.

### DETAILED DESCRIPTION OF ILLUSTRATIVE EMBODIMENTS

Hereinafter, an embodiment of the present invention will be described with reference to the accompanying drawings. The present invention is not limited to the following embodiment of the invention.

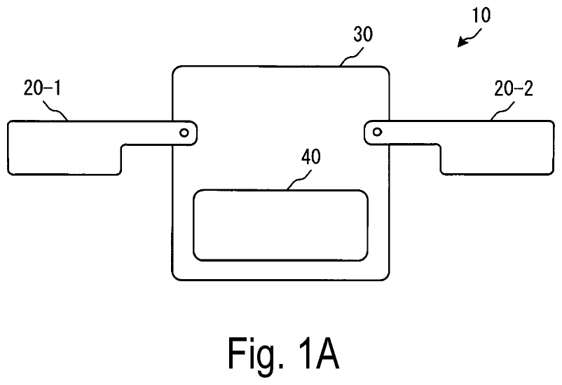

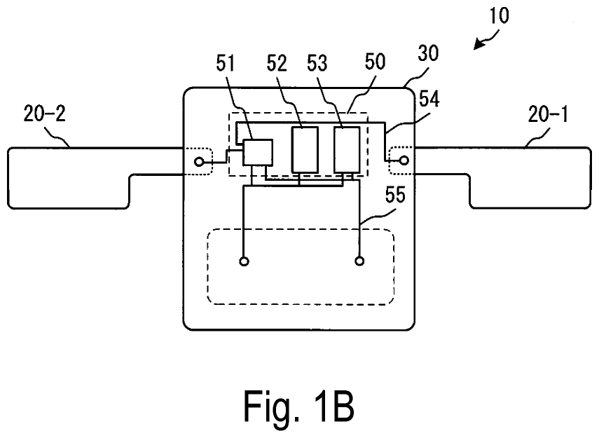

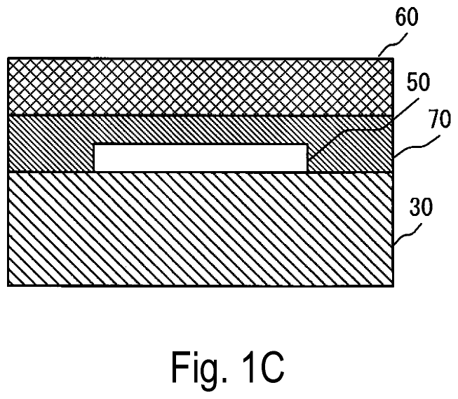

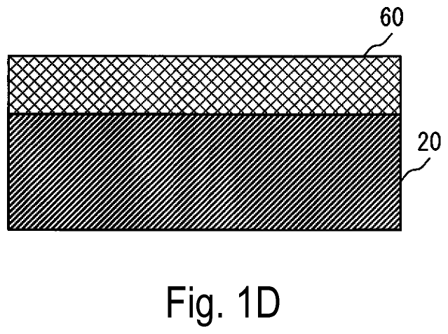

A biometric sensor according to the embodiment of the present invention is a biometric sensor that acquires biometric information of a living body by adhering to clothes to be mounted on the living body. FIGS. 1A and 1B illustrate configuration examples of the biometric sensor according to the embodiment of the present invention. FIG. 1A illustrates a configuration example of a surface (front surface) of a biometric sensor 10 that does not adhere to clothes, and FIG. 1B illustrates a configuration example of a surface (rear surface) of the biometric sensor 10 that adheres to the clothes. The biometric sensor 10 according to the present embodiment includes electrodes (20-1, 20-2) that detect a biometric signal and a substrate 30 to which the electrodes (20-1, 20-2) are connected. Meanwhile, in the following description, an electrocardiac signal or the like which is detected by the electrodes is referred to as a biometric signal, and information such as an electrocardiogram or a heartbeat extracted by processing the detected biometric signal is referred to as biometric information.

The electrodes (20-1, 20-2) that detect the biometric signal can be formed from a silver fabric or a conductive fabric such as hitoe. A fabric substrate can also be used as the substrate. By making the electrodes and the substrate made of fabric, even in a case where the shape of the clothes to which the biometric sensor 10 adheres is deformed in accordance with the form of a living body, the shapes of the electrodes (20-1, 20-2) and the substrate 30 change in accordance with the deformation of the shape of the clothes. Thus, it is possible to increase the degree of intimate contact of the electrodes to the living body while reducing discomfort that the living body feels when the electrodes are mounted.

A signal processing unit 50, a first wiring 54, a battery 40, and a second wiring 55 are disposed on the substrate 30. The signal processing unit 50 processes a biometric signal detected by the electrodes (20-1, 20-2) and extracts biometric information such as an electrocardiogram or a heartbeat. The first wiring 54 sends the biometric signal detected by the electrodes (20-1, 20-2) to the signal processing unit 50. The battery 40 supplies power to the signal processing unit 50. The second wiring 55 supplies power.

The signal processing unit 50 includes an analog front end 51, a CPU 52, and a wireless communication module 53. The analog front end 51 amplifies a signal for capturing weak potential fluctuation of a biometric signal. The CPU 52 performs signal processing such as digitization, filtering, or heartbeat extraction on the signal from the analog front end 51. The wireless communication module 53 transmits, for example wirelessly, biometric information such as an electrocardiogram or a heart rate obtained by the CPU 52. It is also possible to use a CPU module in which the CPU 52 and the wireless communication module 53 are formed integrally with each other.

In addition, it is also possible to acquire more information relating to a living body by mounting an acceleration sensor, a gyro sensor, an orientation sensor, an air pressure sensor, a temperature and humidity sensor, or the like in addition to the analog front end 51.

The biometric sensor of the present embodiment adheres to clothes using an adhesive member 60 provided on the surfaces of the electrodes (20-1, 20-2) and the substrate 30 that adhere to the clothes. FIGS. 1C and 1D are cross-sectional views of the portion of the substrate 30 and the portions of the electrodes (20-1, 20-2) of the biometric sensor to according to the present embodiment. The circuit of the signal processing unit 50 such as the CPU 52 disposed on the substrate 30 is disposed in a waterproof member 70 located on the substrate 30 so as to be protected from moisture such as sweat.

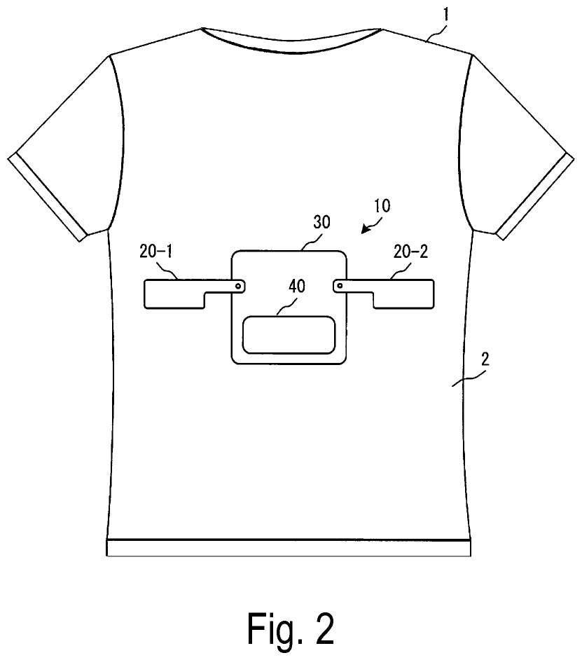

An adhesive agent made of a thermosetting resin or a thermoplastic resin or a removable double-sided adhesive tape can be used as the adhesive member 60. FIG. 2 illustrates a configuration example of clothes to which the biometric sensor according to the present embodiment adheres. In a case where an adhesive agent made of a thermosetting resin or a thermoplastic resin is used as the adhesive member 60, the adhesive agent is applied to the surfaces of the electrodes (20-1, 20-2) and the substrate 30 that adhere to clothes 1, the biometric sensor 10 is placed on a rear surface 2 of the clothes 1 and can adhere to the clothes 1 by for example ironing.

Here, the adhesive member such as an adhesive agent is only required for the biometric sensor 10 to be fixed to the clothes 1 and does not need to be provided on the entire surfaces (rear surfaces) of the electrodes (20-1, 20-2) and the substrate 30 that adhere to the clothes. The adhesive member may be configured to be provided on parts of the rear surfaces of the electrodes and the substrate. For example, there is also a case where it is only required that the electrodes and the substrate partially adhere to the clothes, or a case where it is only required that the substrate adhere to the clothes with the adhesive agent applied only to the rear surface of the substrate in a configuration in which most of the electrodes are disposed on the substrate.

In this manner, the biometric sensor of the present embodiment is configured to include the adhesive member for adhering to the clothes on the surfaces of the electrodes and the substrate that adhere to the clothes, so that a simple method of making the biometric sensor adhere allows clothes selected by a user to measure an electrocardiogram or a heartbeat.

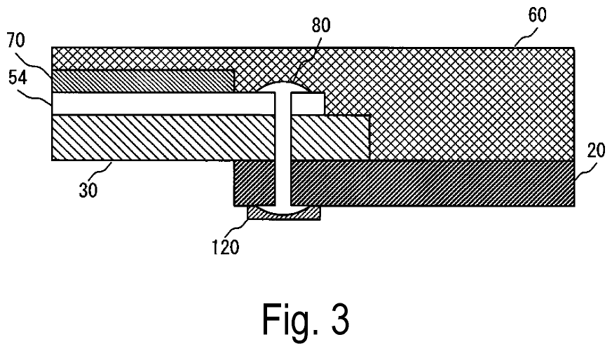

For the connection between the substrate 30 and the electrode 20, the first wiring 54 of the substrate 30 and the electrode 20 may be sewn together using conductive silver thread or the like, or a conductive terminal may be driven using an eyelet 80 or the like as illustrated in FIG. 3. In a case where the eyelet 80 is used, the surface of the electrode 20 comes into contact with the skin, and thus the portion of the eyelet 80 on the surface of the electrode 20 may be covered with a protective sheet 120, a hot melt, or the like.

Either a primary battery or a secondary battery can be used as the battery 40 that supplies power to the signal processing unit of the substrate 30. For example, in a case where a primary battery is used and battery replacement is made, the battery is disposed on the surface (front surface) of the substrate 30 that does not adhere to the clothes. In a case where a secondary battery is used or a primary battery is used and then disposed, the battery can be mounted on the surface (rear surface) of the substrate 30 that adheres to the clothes. In a case where the biometric sensor 10 itself is disposable, battery replacement is unnecessary, and thus the battery can be mounted on the substrate 30 by the same method as electronic parts such as the CPU 52.

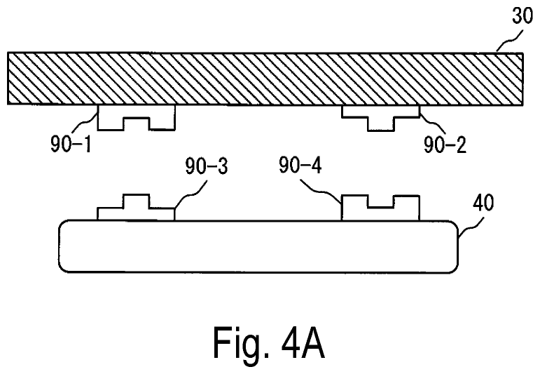

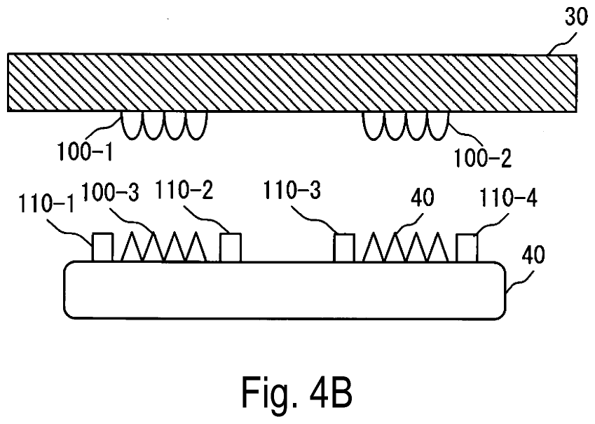

In a case where a primary battery is used as the battery 40 and the battery 40 is replaced, the battery is disposed on the front surface of the substrate 30 which does not adhere to the clothes. FIGS. 4A and 4B illustrate configuration examples of an engagement portion between the substrate 30 and the battery 40. Conductive snap buttons (90-1 to 90-4) or conductive hook and loop fasteners (100-1 to 100-4) are used in the engagement portion between the substrate 30 and the battery 40, so that it is possible to replace the battery while preventing the battery from falling off.

In a case where the snap buttons are used for the engagement between the substrate 30 and the battery 40 of the biometric sensor, it is possible to avoid the risk of erroneously connecting the positive electrode and the negative electrode of the battery 40 by making the disposition relation between the male snap button (90-3) and the female snap button (90-1) opposite to the disposition relation between the male snap button (90-2) and the female snap button (90-4) as illustrated in FIG. 4A.

In a case where the snap buttons are used in the engagement portion between the substrate 30 and the battery 40, wearing the clothes with the battery 40 removed is likely to cause the snap buttons to damage a wearer's skin. In this case, as illustrated in FIG. 4B, providing the female parts (100-1, 100-2) (soft parts) of the conductive hook and loop fasteners on the substrate 30 prevents damage to the skin even when the clothes are worn with the battery 40 removed.

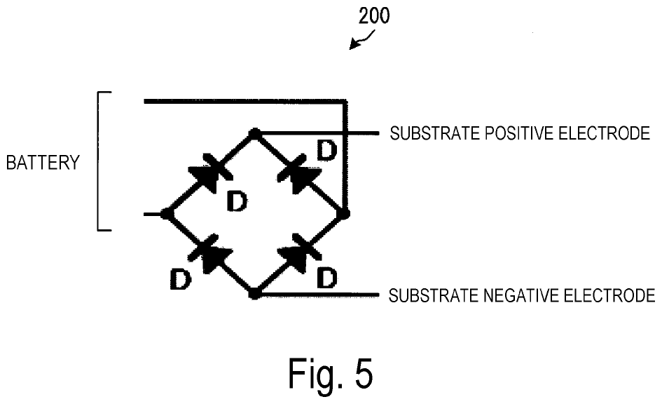

In a case where the hook and loop fasteners (100-1 to 100-4) are used in the engagement portion between the substrate 30 and the battery 40, there is the risk of erroneously connecting the positive electrode and the negative electrode of the battery 40. In order to prevent this, it is only required that a bridge rectifier circuit 200 as illustrated in FIG. 5 be installed between the substrate and the power supply.

In addition, in a case where the hook and loop fasteners and the like are exposed to the engagement portion of the battery 40 and placed on a metal plate or the like, the electrodes of the battery 40 are likely to be short-circuited. In order to prevent such a short circuit between the electrodes, it is only required that both ends of the hook and loop fastener and the like be provided with insulating walls (110-1 to 110-4), and that the battery 40 be provided with an overcurrent protection circuit.

### INDUSTRIAL APPLICABILITY

The embodiments of the present invention can be used as a wearable biometric sensor which is used for routinely acquiring a biometric signal such as an electrocardiac signal.

### REFERENCE SIGNS LIST

10 Biometric sensor
20, 20-1, 20-2 Electrode
30 Substrate
40 Battery
50 Signal processing unit
51 Analog front end
52 CPU
53 Wireless communication module
54 First wiring
55 Second wiring
60 Adhesive member
70 Waterproof member

1.-8. (canceled)

9. A biometric sensor comprising:
a substrate;
an electrode connected to the substrate, the electrode configured to detect a biometric signal from a living body;
a signal processor mounted on the substrate, the signal processor configured to process the biometric signal detected by the electrode to extract biometric information of the living body;
a first wiring mounted on the substrate, the first wiring configured to send the biometric signal detected by the electrode to the signal processor;
a power supply mounted on the substrate, the power supply configured to supply power to the signal processor;
a second wiring mounted on the substrate, the second wiring configured to supply the power from the power supply to the signal processor; and
an adhesive member on at least a portion of surfaces of the electrode and the substrate that adhere to clothes to be worn by the living body.

10. The biometric sensor of claim 9, wherein the electrode comprises a conductive fabric, and the substrate is a fabric substrate.

11. The biometric sensor of claim 9, wherein the adhesive member is a double-sided adhesive tape or an adhesive agent.

12. The biometric sensor of claim 9, wherein the power supply comprises a removable battery and is mounted on a surface of the substrate that does not adhere to the clothes.

13. The biometric sensor of claim 12, wherein the substrate comprises a first engagement portion and a second engagement portion on a surface of the substrate opposite to the power supply, wherein the power supply comprises a third engagement portion and a fourth engagement portion on a surface opposite to the substrate so as to be engaged with the first engagement portion and the second engagement portion, respectively, and wherein the first engagement portion, the second engagement portion, the third engagement portion, and the fourth engagement portion have conductivity.

14. The biometric sensor of claim 13, wherein the first engagement portion and the fourth engagement portion are female snap buttons, and wherein the second engagement portion and the third engagement portion are male snap buttons.

15. The biometric sensor of claim 13, wherein the first engagement portion and the second engagement portion are female parts of hook and loop fasteners, and the third engagement portion and the fourth engagement portion are male parts of the hook and loop fasteners.

16. The biometric sensor of claim 15, further comprising a bridge rectifier circuit between the substrate and the power supply.

17. A biometric sensor comprising:
a fabric substrate;
an electrode on a front surface of the fabric substrate, the electrode configured to detect a biometric signal from a living body;
an adhesive on a rear surface of the electrode, a sidewall of the fabric substrate, and a rear surface of the fabric substrate;
a waterproof member between the adhesive and the fabric substrate; and
a signal processor in the waterproof member, the signal processor configured to process the biometric signal detected by the electrode, and to extract biometric information of the living body.

18. The biometric sensor of claim 17, wherein the biometric information is an electrocardiogram of the living body.

19. The biometric sensor of claim 17, wherein the biometric information is a heartbeat of the living body.

20. The biometric sensor of claim 17, wherein the adhesive is a thermosetting resin.

21. The biometric sensor of claim 17, wherein the adhesive is a thermoplastic resin.

22. The biometric sensor of claim 17, wherein the adhesive is a double-sided adhesive.

23. The biometric sensor of claim 17 further comprising:
wiring mounted on the fabric substrate, the wiring connecting the electrode to the signal processor, the adhesive further disposed on a rear surface of the wiring.

24. The biometric sensor of claim 23 further comprising:
an eyelet extending through the wiring, the fabric substrate, and the electrode.

25. The biometric sensor of claim 17 further comprising:
a battery on the front surface of the fabric substrate, the battery configured to supply power to the signal processor.

26. The biometric sensor of claim 25 further comprising:
conductive snap buttons connecting the battery to the fabric substrate.

27. The biometric sensor of claim 25 further comprising:
conductive hook and loop fasteners connecting the battery to the fabric substrate.
